(19) United States
(12) Patent Application Publication
(10) Pub. No.: US 2021/0353878 A1
(43) Pub. Date: Nov. 18, 2021

(54) HYPODERMIC DEVICE WITH AN ARRAY OF HYPODERMIC NEEDLES

(71) Applicant: Jonathan Robin Fugo, Wappingers Falls, NY (US)

(72) Inventor: Jonathan Robin Fugo, Wappingers Falls, NY (US)

(21) Appl. No.: 15/931,631

(22) Filed: May 14, 2020

(51) Int. Cl. A61M 5/32; A61M 39/10

(52) U.S. Cl. CPC A61M 5/3286; A61M 2039/1033; A61M 39/10

(57) ABSTRACT

A hypodermic device is disclosed comprising a chamber, an array of hypodermic needles and a threaded Luer lock connector. The chamber comprises a distal outer surface and a proximal outer surface. The array of hypodermic needles projects outward from the distal outer surface of the chamber. Each hypodermic needle comprises a bore extending from a receiving end of the hypodermic needle to a delivery end of the hypodermic needle along a longitudinal axis. The bore in each of the hypodermic needles is in fluid communication with a hollow space in the chamber. The threaded Luer lock connector is in fluid communication with an opening at the proximal outer surface of the chamber. A first end of the threaded Luer lock connector is connected to the proximal outer surface of the chamber.

### BACKGROUND

The present invention relates to a hypodermic device for delivering a fluid or a gel to a patient. More specifically, the present invention relates to a hypodermic device comprising an array of hypodermic needles for delivering a precise injection of a fluid or a gel to an intradermal or subdermal tissue with minimal pressure.

Conventional needles create needle phobia, which can often create extreme anxiety, at times resulting in a syncopal episode of the patient, making it necessary to abort the desired injection procedure. Additionally, in order to achieve a desired effect, certain injection procedures demand a pattern of repeated injections into the same, precise soft tissue plane over a large surface area during a single injection session, and/or multiple sessions. In order to overcome these obstacles, needle arrays are used. A needle array device can simultaneously reduce anxiety and deliver repeated injections over a large area into precise soft tissue planes at a consistent, desired depth within or just beneath the skin. Moreover, needle arrays generate less pressure when the patient is injected, thereby resulting in less procedural pain to the patient.

Existing hypodermic needle arrays comprise needle barrels that are open at a first end to receive the injection fluid from a reservoir. The needle barrels are closed at a second end with a sharp tip for penetrating the intradermal or subdermal tissue. The side walls of the needle barrels comprise one or more openings for conveying the injection fluid from within the needle barrels to the intradermal or subdermal tissue that exists between adjacent needle barrels in the array. An example of needle barrels of the hypodermic needle array is shown in FIG. 1. The openings in the side walls are oriented such that the injection fluid is directed through openings in the needles' sidewalls, towards adjacent needles in the array. More specifically, this array is designed to direct injection fluid towards the center of the array, away from the periphery of the device in any plane, circumference or depth. These designs also allow for use of electrodes, coupled within the needle arrays to create an electrical current, imparting an additional, perceived, desired effect into cells of the injected tissue as shown in FIG. 2.

The existing hypodermic needle array design is complicated or inadequate in design primarily for four reasons: (a) the needle barrels are open at the receiving, or proximal end attached to the reservoir, while closed at the opposite or distal end; (b) the needle barrels comprise one or more openings only in the side walls, specifically oriented such that any substance injected through the needles into the soft tissues is directed towards the center of the needle array, providing a specific directionality or field of injection limited to the confines of the perimeter of the needle array, either circumferentially or deep; (c) as the delivery of the injected substance is accomplished only through the sidewall ports in the needle array, delivery of an injected substance to deeper tissue planes necessarily requires passing the tips of the needles within the needle array to greater depths, so as to align the sidewall ports with the desired depth at which the injected substance is to be delivered; (d) the needle barrels are designed for use with electrodes, which can be utilized for directing an electrical current into the soft tissues through which the needle array is being passed.

Therefore, there is a need for a hypodermic device with an array of hypodermic needles which overcomes the deficiencies of the current hypodermic needle array design. Furthermore, there is a need for a hypodermic device with an array of hypodermic needles, where the hypodermic needles in the array comprise an opening at the distal most tip of the hypodermic needles for easily delivering low pressure, repeated, uniform pattern injections to a predetermined, desired, reproducible plane within the intradermal or subdermal soft tissue over a large surface area.

### SUMMARY OF THE INVENTION

This summary is provided to introduce a selection of concepts in a simplified form that are further disclosed in the detailed description of the invention. This summary is not intended to determine the scope of the claimed subject matter.

Disclosed herein is a hypodermic device for delivering an injection of fluid or gel to an intradermal tissue or a subdermal tissue plane. The hypodermic device comprises a chamber, an array of hypodermic needles and a threaded Luer lock connector. The chamber comprises a distal outer surface and a proximal outer surface. The array of hypodermic needles project outward from the distal outer surface of the chamber, approximately perpendicular to the distal outer surface. Each of the hypodermic needles in the array of the hypodermic needles comprises a bore extending from a receiving end of the hypodermic needle to a delivery end of the hypodermic needle along a longitudinal axis. The bore at the receiving end of each of the hypodermic needles is in fluid communication with a hollow space in the chamber. In an embodiment, the diameter of the bore of each of the hypodermic needles is about 27 gauge to about 32 gauge, and the length of each hypodermic needle is about 1 mm to about 10 mm. The threaded Luer lock connector is in fluid communication with an opening at the proximal outer surface of the chamber. A first end of the threaded Luer lock connector is connected to the proximal outer surface of the chamber.

In an embodiment, the chamber comprises a wall extending from the distal outer surface to the proximal outer surface of the chamber, vertically along a circumference of the chamber. In an embodiment, the shape of the chamber extending from the distal outer surface to the proximal outer surface of the chamber is a generally square shape. Furthermore, the length of each side of the generally square shaped chamber is about 2 cm. In an embodiment, the shape of the chamber extending from the distal outer surface to the proximal outer surface of the chamber is a generally triangular shape. In an embodiment, the triangle is an equilateral triangle and the length of each side of the generally triangular shaped chamber is about 2 cm. In an embodiment, the length of each side of the generally square shaped chamber and the generally triangular shaped chamber is about 1 cm to about 5 cm. In another embodiment, the shape of the chamber extending from the distal outer surface to the proximal outer surface of the chamber is one of a generally circular shape, a rectangular shape, and a pentagonal shape.

### BRIEF DESCRIPTION OF DRAWINGS

The foregoing summary, as well as the following detailed description of the invention, is better understood when read in conjunction with the appended drawings. For illustrating the invention, exemplary constructions of the invention are shown in the drawings. However, the invention is not limited to the specific components disclosed herein. The description of a component referenced by a numeral in a drawing is applicable to the description of that component shown by that same numeral in any subsequent drawing herein.

FIG. 1 exemplarily illustrates a set of needle barrels of a needle array with openings in the side walls of the needle barrels, closed at the distal end of the needle barrels.

FIG. 2 exemplarily illustrates electrodes for use with a set of needle barrels of a needle array for introduction of an electrical current into the soft tissues through which the needle array is passed.

FIG. 3 illustrates a front bottom perspective view of an embodiment of a hypodermic device comprising a generally square shaped chamber, an array of hypodermic needles, a threaded Luer lock connector, and a fluid reservoir for delivery of injection fluid to the intradermal or subdermal tissue.

FIG. 4 illustrates a front view of the embodiment of the hypodermic device comprising the generally square shaped chamber.

FIG. 5 illustrates a top view of the embodiment of the hypodermic device comprising the generally square shaped chamber showing the threaded Luer lock connector.

FIG. 6 illustrates a bottom view of the embodiment of a hypodermic device comprising the generally square shaped chamber showing an array of hypodermic needles.

FIG. 7 illustrates a lateral cross-sectional view of the embodiment of the hypodermic device along a line A-A′ in FIG. 6.

FIG. 8 illustrates a front view of an embodiment of the hypodermic device comprising a generally triangle shaped chamber, an array of hypodermic needles, a threaded Luer lock connector, and a fluid reservoir for delivery of injection fluid to an intradermal or subdermal tissue.

FIG. 9 illustrates a top view of the embodiment of the hypodermic device comprising the generally triangle shaped chamber.

FIG. 10 illustrates a bottom view of an embodiment of the hypodermic device comprising the generally triangle shaped chamber with an array of hypodermic needles.

FIG. 11 illustrates a lateral cross-sectional view of the embodiment of the hypodermic device along the line B-B′ in FIG. 10.

### DETAILED DESCRIPTION OF THE INVENTION

The foregoing summary, as well as the following detailed description of the invention, is better understood when read in conjunction with the appended drawings. For illustrating the invention, exemplary constructions of the invention are shown in the drawings. However, the invention is not limited to the specific components disclosed herein. The description of the component referenced by a numeral in a drawing is applicable to the description of that component shown by that same numeral in any subsequent drawing herein.

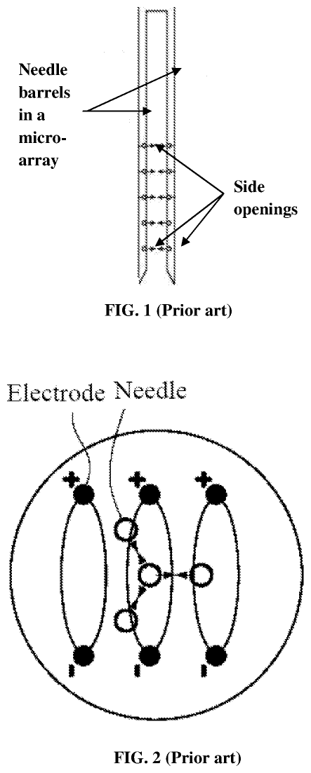

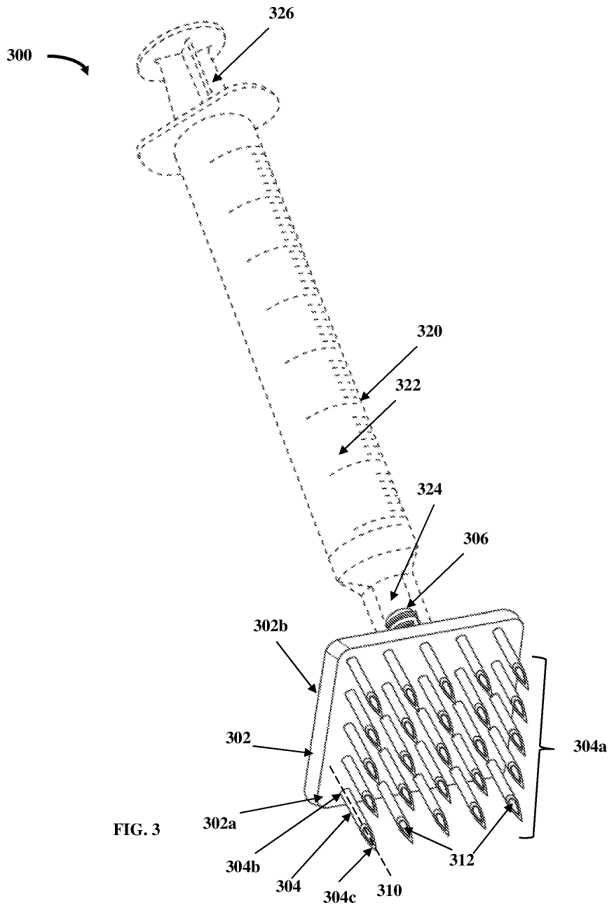

FIG. 1 exemplarily illustrates a prior art set of needle barrels of a needle array. The prior art set of needle barrels comprise openings in the side walls of the needle barrels while the distal end of the needle barrels are closed. FIG. 2 exemplarily illustrates a prior art that uses electrodes in combination with a set of needle barrels of a needle array. The electrodes are used to introduce an electrical current into the soft tissues through which the needle array passes.

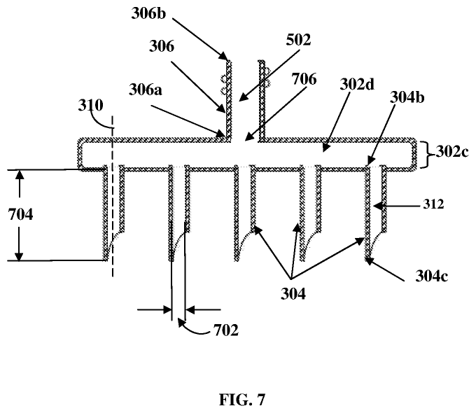

FIG. 3 illustrates a front bottom perspective view of an embodiment of a hypodermic device 300 for delivering an injection fluid to an intradermal tissue or a subdermal tissue plane. The hypodermic device 300 comprises a chamber 302, an array 304a of hypodermic needles 304 and a threaded Luer lock connector 306. The chamber 302 comprises a distal outer surface 302a and a proximal outer surface 302b. The array 304a of hypodermic needles 304 project outward from the distal outer surface 302a of the chamber 302 and substantially perpendicular to the distal outer surface 302a of the chamber 302, as illustrated in FIGS. 3, 4, 6-8 and 10-11. Each of the hypodermic needles 304 in the array 304a comprises a bore 312 extending from a receiving end 304b of the hypodermic needle 304 to a delivery end 304c of the hypodermic needle along a longitudinal axis 310. The bore 312 at the receiving end 304b of each of the hypodermic needles 304 is in fluid communication with a hollow space 302d within the chamber 302. The diameter 702 of the bore 312 of each of the hypodermic needles 304 is about 27 gauge to about 32 gauge, and the length 704 of each hypodermic needle 304 is about 1 mm to about 10 mm, as illustrated in FIGS. 7 and 11. The threaded Luer lock connector 306 is in fluid communication with an opening 706 at the proximal outer surface 302b of the chamber 302. A first end 306a of the threaded Luer lock connector 306 is connected to the proximal outer surface 302b of the chamber 302. The threaded Luer lock connector 306 is located at the center of rotational symmetry of the proximal outer surface 302b of the chamber 302.

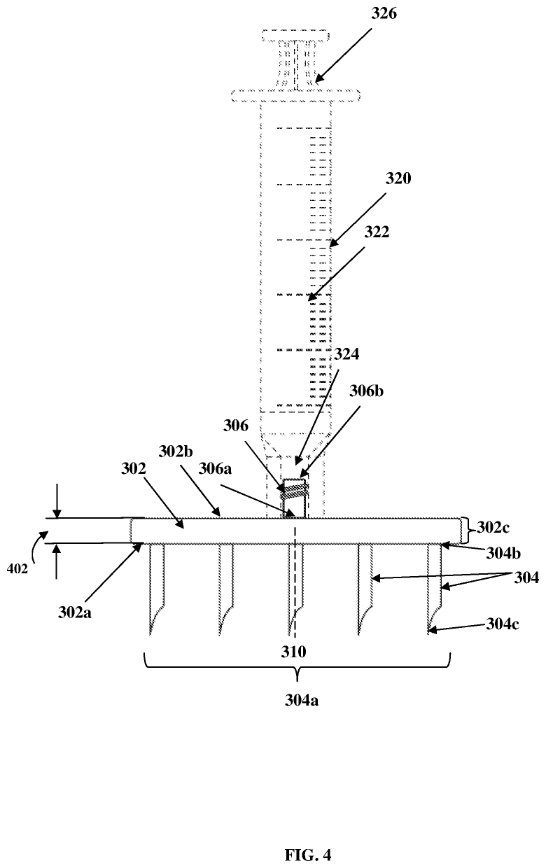

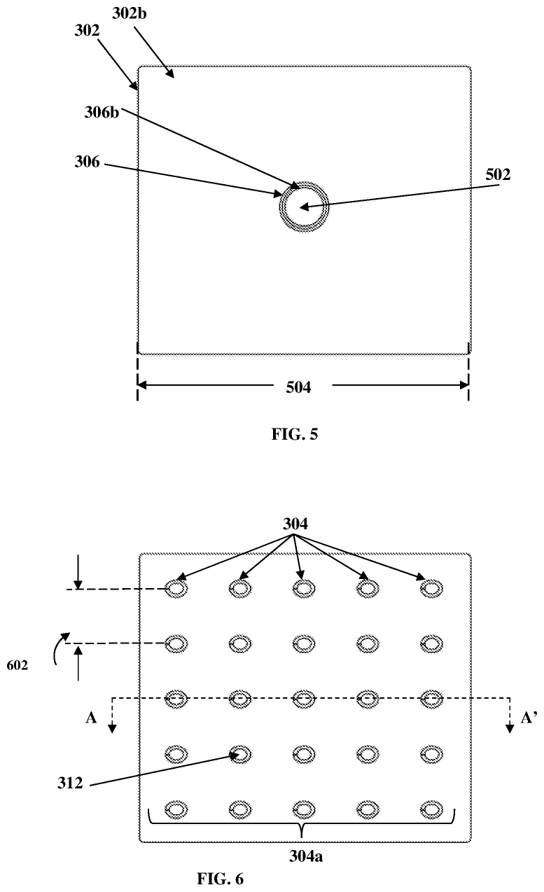

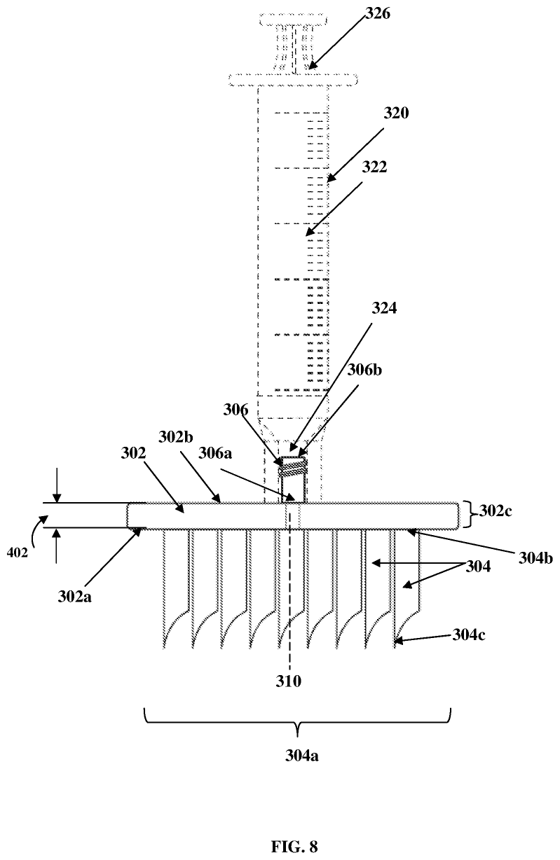

As illustrated in FIGS. 4 and 8, the threaded Luer lock connector 306 comprises the first end 306a and a second end 306b. The second end 306b of the threaded Luer lock connector 306 is configured to mate with a matching Luer fitting 324 of a fluid reservoir 322 of a fluid injection device 320. The fluid reservoir 322 comprises an injection fluid. Furthermore, the fluid reservoir 322 is configured to be pressurized to allow the injection fluid to be forced out of the fluid reservoir 322 through a conduit 502 of the threaded Luer lock connector 306, as illustrated in FIG. 5. The matching Luer fitting 324 is in fluid communication with the fluid reservoir 322. The fluid reservoir 322 is configured to deliver the injection fluid to the chamber 302 through the matching Luer fitting 324 and the threaded Luer lock connector 306 when the fluid reservoir 322 is pressurized. In an embodiment, the fluid reservoir 322 is the fluid reservoir of a syringe 320, as illustrated in FIGS. 3-4 and 8.

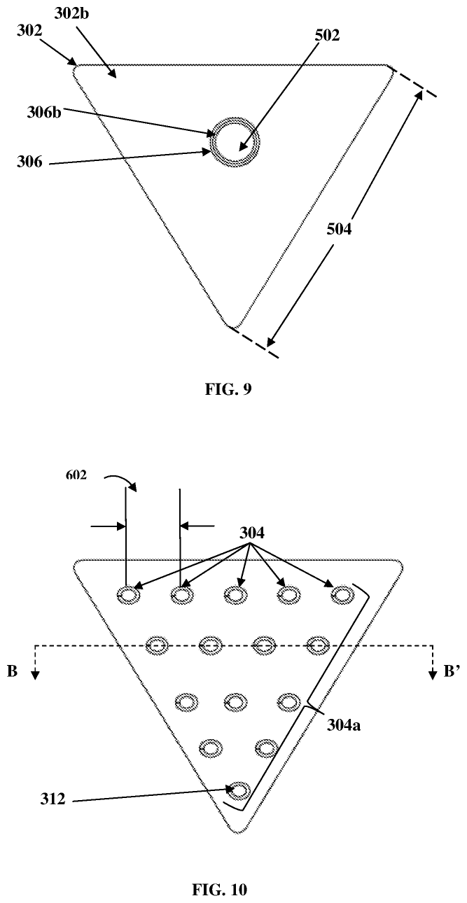

In an embodiment, the chamber 302 comprises a wall 302c extending from the distal outer surface 302a to the proximal outer surface 302b of the chamber. In an embodiment, the wall 302c extends vertically from a circumference of the chamber 302. In an embodiment, the shape of the chamber 302 extending from the distal outer surface 302a to the proximal outer surface 302b of the chamber 302 is a generally square shape, as illustrated in FIGS. 3-7. As illustrated in FIGS. 5 and 9, the length 504 of each side of the square shaped chamber 302 is about 2 cm. In an embodiment, the length 504 of each side of the square shaped chamber 302 is about 1 cm to about 5 cm.

FIG. 4 illustrates a front view of the embodiment of the hypodermic device 300 comprising the generally square shaped chamber 302. As illustrated in FIG. 4, thickness 402 of the chamber 302 extending from the distal outer surface 302a to the proximal outer surface 302b is about 0.2 cm to about 2 cm. In an embodiment, the thickness 402 of the chamber 302 extending from the distal outer surface 302a to the proximal outer surface 302b is about 0.1 cm to about 2 cm.

FIG. 5 illustrates a top view of the embodiment of the hypodermic device 300 comprising the generally square shaped chamber 302 and the threaded Luer lock connector 306.

FIG. 6 illustrates a bottom view of the embodiment of a hypodermic device 300 comprising the generally square shaped chamber 300 showing the array 304a of hypodermic needles 304.

FIG. 7 illustrates a lateral cross-sectional view of the embodiment of the hypodermic device 300 along a line A-A′ in FIG. 6.

FIG. 8 illustrates a front view of the embodiment of the hypodermic device 300 comprising the generally triangle shaped chamber 302. As illustrated in FIG. 8, the thickness 402 of the chamber 302 extending from the distal outer surface 302a to the proximal outer surface 302b is about 0.1 cm to about 2 cm. In an embodiment, the thickness 402 of the chamber 302 extending from the distal outer surface 302a to the proximal outer surface 302b is about 0.2 cm to about 2 cm.

In an embodiment, illustrated in FIGS. 8-11, the shape of the chamber 302 extending from the distal outer surface 302a to the proximal outer surface 302b of the chamber 302 is generally triangle shaped. In an embodiment, the shape of the chamber 302 extending from the distal outer surface 302a to the proximal outer surface 302b of the chamber 302 is generally an equilateral triangle shape. The generally equilateral triangle shape of the chamber 302 allows for a uniform spaced pattern of subsequent placements of the needle array 300 along arcuate or otherwise non-linear paths, without overlapping previous needle array 300 placements, nor leaving significant gaps between subsequent needle array 300 placements. The length 502 of each side of the generally triangle shaped chamber 302 is about 2 cm. In an embodiment, the length 502 of each side of the generally triangular shaped chamber 302 is about 1 cm to about 5 cm.

FIG. 9 illustrates a top view of the embodiment of the hypodermic device 300 comprising the generally triangle shaped chamber 302 and the threaded Luer lock connector 306. In an embodiment, the shape of the chamber 302 extending from the distal outer surface 302a to the proximal outer surface 302b of the chamber 302 is one of a generally circular shape, a generally rectangular shape, and a generally pentagonal shape, a polygonal shape, etc.

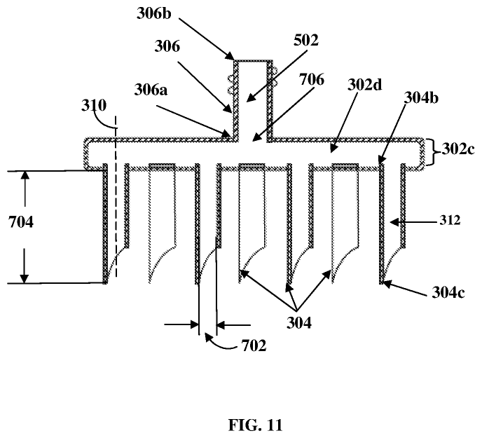

FIG. 10 illustrates a bottom view of the embodiment of the hypodermic device comprising the generally triangle shaped chamber 302 showing the array 304a of hypodermic needles 304.

FIG. 11 illustrates a lateral cross-sectional view of the embodiment of the hypodermic device 300 along the line B-B′ in FIG. 10, comprising the generally triangle shaped chamber 302 and the threaded Luer lock connector 306.

As illustrated in FIGS. 7 and 11, the threaded Luer lock connector 306 comprises a conduit 502 for the delivery of the injection fluid from the pressurized fluid reservoir 322 to the chamber 302. The conduit 502 is in fluid communication with an opening 706 at the proximal outer surface 302b of the chamber 302. In an embodiment, the opening 706 at the proximal outer surface 302b of the chamber 302 is located at either a center of rotational symmetry or a centroid of the chamber 302 at the proximal outer surface 302b of the chamber 302.

As shown in FIGS. 6 and 10, the hypodermic needles 304 are arranged symmetrically within the array 304a of hypodermic needles 304. The distance 602 between adjacent hypodermic needles 304 in the array 304a of hypodermic needles 304 is about 1 mm to about 10 mm.

When pressure is applied to the fluid reservoir 322, for example, a syringe 320 by pressing a plunger 326, the injection fluid in the now pressurized fluid reservoir 322 enters the conduit 502 of the chamber 302. From the conduit 502, the injection fluid enters the opening 706 and passes into the hollow space 302d of the chamber 302. The injection fluid fills the chamber 302 and enters the bore 312 at the receiving end 304b of each of the hypodermic needles 304 in the array 304a of hypodermic needles 304. The injection fluid fills the bore 312 of the hypodermic needles 304 from the chamber 302 which is oriented at about 90 degrees with respect to the longitudinal axis 310 of each of the hypodermic needles 304. The injection fluid exits the bore 312 at the delivery end 304c of each of the hypodermic needles 304 in the array 304a of hypodermic needles 304 into the intradermal tissue or the subdermal tissue at about 90 degrees with respect to the longitudinal axis 310 of the hypodermic needles 304.

In an embodiment, the array 304a comprises about three hypodermic needles 304, arranged symmetrically in a generally triangular shaped chamber 302. In another embodiment, the hypodermic needles 304 comprise about four hypodermic needles 304, arranged symmetrically in a generally square shaped chamber 302. In an embodiment, the chamber 302 and the array 304a of hypodermic needles 304 are fabricated by tube drawing, extrusion molding, injection molding, or a combination thereof. In an embodiment, the maximum number of hypodermic needles 304 in the array 304a is 256.

The hypodermic device 300 with the array 304a of hypodermic needles 304 overcomes several drawbacks in the existing hypodermic device with a needle array design. For example, the needle array in the existing hypodermic device exemplarily illustrated in FIG. 1 comprises needle barrels that are open at the proximal receiving end to receive the injection fluid from the pressurized fluid connector, and are closed at the distal end. This distal, closed end has a sharp tip for penetrating the intradermal or subdermal tissue. Dispensing of the injected fluid is accomplished only through ports created in the sidewalls of the needle barrels, as the side walls of the needle barrels in the existing hypodermic needle array comprise one or more openings designed for delivering the injection fluid from the needle barrels to the intradermal or subdermal tissue between adjacent needle barrels. Furthermore, in the existing hypodermic needle array design, openings in the side walls are specifically oriented such that the injected fluid is directed towards adjacent needles in the array and simultaneously towards the center of the needle array, away from the peripheral margins of the array, either circumferentially or deep as exemplarily illustrated in FIG. 2. Additionally, existing hypodermic arrays are outfitted with electrodes that can be utilized to convey an electrical current along the needle barrels, thereby directing said current into the soft tissues through which the needle array is being passed.

In contrast, the hypodermic device 300 with the array 304a of hypodermic needles 304 illustrated in FIGS. 3-11 is structurally simplified. It employs the conventional structure for hypodermic needles 304 with needle barrels 312 open at both the receiving end 304b and delivery end 304c, but with no openings in the barrel 312 sidewalls, with the hypodermic needles 304 having a length in the millimeter to centimeter range. Furthermore, in contrast to the needle array in the existing hypodermic array design, the hypodermic device 300 with the array 304a of hypodermic needles 304 comprises hypodermic needles 304 having a barrel 312 that is open only at the receiving end 304b and delivery end 304c of the hypodermic needle 304 to receive the injection fluid from the fluid reservoir 322 of the fluid injection device 320, and thereafter deliver the injection fluid to the intradermal tissue or the subdermal tissue. Therefore, the hypodermic device 300 with the array 304a of hypodermic needles 304 overcomes the problem of an injection delivery limited to the area of the array 304a defined by the directionality of sidewall ports. Furthermore, the hypodermic device 300 with the array 304a of hypodermic needles 304 delivers the injection fluid only through the delivery end 304c, thereby overcoming the need to pass the needles 304 deeper than the desired soft tissue plane of injected fluid delivery. Use of the existing hypodermic needle array design necessarily demands a needle penetration depth greater than the desired plane of injected fluid delivery so as to align the sidewall ports with the desired depth at which the injected substance is to be delivered. Furthermore, the hypodermic device 300 with the array 304a of hypodermic needles 304 also overcomes the need for electrodes within the needle barrels 312 as there is no role for electrical current in the hypodermic device 300 with the array 304a of hypodermic needles 304. Furthermore, since the array 304a of hypodermic needles 304 is generally symmetric, it can be easily configured and fabricated through any one or a combination of traditional, readily available fabrication methods, for example by tube drawing, extension molding, or injection molding, even when the diameter 702 of the bore 312 of the hypodermic needles 304 is in the micrometer range. Moreover, the direct delivery of injection fluid into the desired intradermal or subdermal tissue planes through the delivery end 304c of the hypodermic needle 304 rather than via the openings in the side walls of the needle barrels 312 makes it easier to maintain the desired low pressure applied to the fluid connector 320. Furthermore, the delivery of injected substance through the delivery end 304c of the hypodermic needle 304 alleviates the number of injections to be done, as the injected substance has no directionality beyond that outward from the distal end of the needle 304, and thus may spread freely and equally about the distal needle site proximal to the delivery end 304c.

The foregoing examples have been provided merely for explanation and are in no way to be construed as limiting of the hypodermic device 300 with the array 304a of hypodermic needles 304 disclosed herein. While the hypodermic device 300 with the array 304a of hypodermic needles 304 has been described with reference to a particular embodiment, it is understood that the words, which have been used herein, are words of description and illustration, rather than words of limitation. Furthermore, although the hypodermic device 300 with the array 304a of hypodermic needles 304 has been described herein with reference to a particular means, materials, and embodiment, the hypodermic device 300 with the array 304a of hypodermic needles 304 is not intended to be limited to the particulars disclosed herein; rather, the design and functionality of the hypodermic device 300 with the array 304a of hypodermic needles 304 extends to all functionally equivalent structures and uses, such as are within the scope of the appended claims. Furthermore, it will be understood by those skilled in the art, having the benefit of the teachings of this specification, that the hypodermic device 300 with the array 304a of hypodermic needles 304 disclosed herein is capable of modifications and other embodiments may be effected and changes may be made thereto, without departing from the scope and spirit of the hypodermic device 300 with the array 304a of hypodermic needles 304 disclosed herein.

I claim:

1. A hypodermic device, comprising:
a chamber comprising a distal outer surface and a proximal outer surface;
an array of hypodermic needles projecting outward from the distal outer surface of the chamber and perpendicular to the distal outer surface, wherein each of the hypodermic needles comprises a bore extending from a receiving end of the hypodermic needle to a delivery end of the hypodermic needle along a longitudinal axis, wherein the bore at the receiving end of each of the hypodermic needles is in fluid communication with a hollow space in the chamber, wherein diameter of the bore of each of the hypodermic needles is about 27 gauge to about 32 gauge, and wherein a length of each hypodermic needle is about 1 mm to about 10 mm; and
a threaded Luer lock connector in fluid communication with an opening at the proximal outer surface of the chamber, wherein a first end of the threaded Luer lock connector is connected to the proximal outer surface of the chamber.

2. The hypodermic device of claim 1, wherein a second end of the threaded Luer lock connector is configured to mate with a matching Luer fitting of a fluid reservoir.

3. The hypodermic device of claim 2, wherein the fluid reservoir comprises an injection fluid, wherein the fluid reservoir is configured to be pressurized, wherein the matching Luer fitting is in fluid communication with the fluid reservoir, and wherein the fluid reservoir is configured to deliver the injection fluid to the chamber through the matching Luer fitting and the threaded Luer lock connector when the fluid reservoir is pressurized.

4. The hypodermic device of claim 3, wherein the fluid reservoir that is configured to be pressurized is a syringe.

5. The hypodermic device of claim 3, wherein the threaded Luer lock connector comprises a conduit for the delivery of the injection fluid from the pressurized fluid reservoir to the chamber.

6. The hypodermic device of claim 3, wherein the chamber is oriented at about 90 degrees with respect to the longitudinal axis of each of the hypodermic needles, wherein the injection fluid enters the bore at the receiving end of each of the hypodermic needles in the array of hypodermic needles from the chamber, and wherein the injection fluid exits the bore at the delivery end of each of the hypodermic needles in the array of hypodermic needles directly into an intradermal tissue or a subdermal tissue at 90 degrees with respect to the longitudinal axis of each of the hypodermic needles.

7. The hypodermic device of claim 1, wherein the chamber comprises a wall extending from the distal outer surface to the proximal outer surface, and wherein a shape of the chamber extending from the distal outer surface to the proximal outer surface the chamber is one of a generally square shape and a generally triangular shape.

8. The hypodermic device of claim 7, wherein a length of each side of the generally square shaped chamber and the generally triangular shaped chamber is about 2 cm.

9. The hypodermic device of claim 7, wherein a length of each side of the generally square shaped chamber and the generally triangular shaped chamber is about 1 cm to about 5 cm.

10. The hypodermic device of claim 1, wherein a shape of the chamber extending from the distal outer surface to the proximal outer surface of the chamber is one of a generally circular shape, a generally rectangular shape, and a generally pentagonal shape.

11. The hypodermic device of claim 1, wherein a distance between adjacent hypodermic needles in the array of hypodermic needles is about 1 mm to about 10 mm.

12. The hypodermic device of claim 1, wherein the opening at the proximal outer surface of the chamber is located at one of a center of rotational symmetry and a centroid of the chamber at the proximal outer surface of the chamber.

13. The hypodermic device of claim 1, wherein the hypodermic needles are arranged symmetrically within the array of hypodermic needles.
